(19) United States
(12) Patent Application Publication
(10) Pub. No.: US 2008/0161809 A1
(43) Pub. Date: Jul. 3, 2008

(54) Articulating Tissue Cutting Device

(75) Inventors: Gregory Schmitz, Los Gatos, CA (US); Jeffery L. Bleich, Palo Alto, CA (US); Eric C. Miller, Los Gatos, CA (US)

Correspondence Address: SHAY GLENN LLP, 2755 CAMPUS DRIVE, SUITE 210, SAN MATEO, CA, 94403, US

(73) Assignee: BAXANO, INC., Mountain View, CA (US)

(21) Appl. No.: 11/538,345

(22) Filed: Oct. 3, 2006

(51) Int. Cl. A61B 17/00

(52) U.S. Cl. 606/79

(57) ABSTRACT

A device for cutting ligament and/or bone tissue in a lateral recess and/or an intervertebral foramen of a spine of a patient to treat spinal stenosis may include: an elongate shaft having a rigid proximal portion and a distal portion articulatable relative to the proximal portion; a handle coupled with the proximal portion of the shaft; a tissue cutter disposed on one side of the distal portion of the shaft; a first actuator coupling the handle with the tissue cutter for activating the tissue cutter to cut tissue; and a second actuator coupling the handle with the distal portion for articulating the distal portion relative to the proximal portion. In some embodiments, the distal portion of the shaft may be configured to pass at least partway into an intervertebral foramen of the patient's spine.

### BACKGROUND OF THE INVENTION

The present invention relates generally to medical/surgical devices and methods. More specifically, the present invention relates to a tissue cutting devices and methods.

A significant number of surgical procedures involve cutting, shaving, abrading or otherwise contouring or modifying tissue in a patient's body. As the demand for less invasive surgical procedures continually increases, performing various tissue modifications such as cutting, contouring and removing tissue often becomes more challenging. Some of the challenges of minimally invasive procedures include working in a smaller operating field, working with smaller devices, and trying to operate with reduced or even no direct visualization of the structure (or structures) being treated. For example, using arthroscopic surgical techniques for repairing joints such as the knee or the shoulder, it may be quite challenging to cut certain tissues to achieve a desired result, due to the required small size of arthroscopic instruments, the confined surgical space of the joint, lack of direct visualization of the surgical space, and the like. It may be particularly challenging in some surgical procedures, for example, to cut or contour bone or ligamentous tissue with currently available minimally invasive tools and techniques. For example, trying to shave a thin slice of bone off a curved bony surface, using a small-diameter tool in a confined space with little or no ability to see the surface being cut, as may be required in some procedures, may be incredibly challenging or even impossible using currently available devices.

Examples of less invasive surgical procedures include laparoscopic procedures, arthroscopic procedures, and minimally invasive approaches to spinal surgery, such as a number of less invasive intervertebral disc removal, repair and replacement techniques. One area of spinal surgery in which a number of less invasive techniques have been developed is the treatment of spinal stenosis. Spinal stenosis occurs when one or more tissues in the spine impinges upon neural and/or neurovascular tissue, causing symptoms such as lower limb weakness, numbness and/or pain. This impingement of tissue may occur in one or more of several different areas in the spine, such as in the central spinal canal, or more commonly in the lateral recesses of the spinal canal and/or one or more intervertebral foramina.

FIGS. 1-3 show various partial views of the lower (lumbar) region of the spine. FIG. 1 shows an approximate top view of a vertebra with the cauda equina (the bundle of nerves that extends from the base of the spinal cord through the central spinal canal) shown in cross section and two nerve roots exiting the central spinal canal and extending through intervertebral foramina on either side of the vertebra. The spinal cord and cauda equina run vertically along the spine through the central spinal canal, while nerve roots branch off of the spinal cord and cauda equina between adjacent vertebrae and extend through the intervertebral foramina. Intervertebral foramina may also be seen in FIGS. 2 and 3, and nerves extending through the foramina may be seen in FIG. 2.

One common cause of spinal stenosis is buckling and thickening of the ligamentum flavum (one of the ligaments attached to and connecting the vertebrae), as shown in FIG. 1. (Normal ligamentum flavum is shown in cross section in FIG. 3) Buckling or thickening of the ligamentum flavum may impinge on one or more neurovascular structures, dorsal root ganglia, nerve roots and/or the spinal cord itself. Another common cause of neural and neurovascular impingement in the spine is hypertrophy of one or more facet joints (or “zygopophaseal joints”), which provide articulation between adjacent vertebrae. (Two vertebral facet superior articular processes are shown in FIG. 1. Each superior articular process articulates with an inferior articular process of an adjacent vertebra to form a zygopophaseal joint. Such a joint is labeled in FIG. 3.) Other causes of spinal stenosis include formation of osteophytes (or “bone spurs”) on vertebrae, spondylolisthesis (sliding of one vertebra relative to an adjacent vertebra), facet joint synovial cysts, and collapse, bulging or herniation of an intervertebral disc into the central spinal canal. Disc, bone, ligament or other tissue may impinge on the spinal cord, the cauda equina, branching spinal nerve roots and/or blood vessels in the spine to cause loss of function, ischemia and even permanent damage of neural or neurovascular tissue. In a patient, this may manifest as pain, impaired sensation and/or loss of strength or mobility.

In the United States, spinal stenosis occurs with an incidence of between 4% and 6% of adults aged 50 and older and is the most frequent reason cited for back surgery in patients aged 60 and older. Conservative approaches to the treatment of symptoms of spinal stenosis include systemic medications and physical therapy. Epidural steroid injections may also be utilized, but they do not provide long lasting benefits. When these approaches are inadequate, current treatment for spinal stenosis is generally limited to invasive surgical procedures to remove ligament, cartilage, bone spurs, synovial cysts, cartilage, and bone to provide increased room for neural and neurovascular tissue. The standard surgical procedure for spinal stenosis treatment includes laminectomy (complete removal of the lamina (see FIGS. 1 and 2) of one or more vertebrae) or laminotomy (partial removal of the lamina), followed by removal (or “resection”) of the ligamentum flavum. In addition, the surgery often includes partial or occasionally complete facetectomy (removal of all or part of one or more facet joints). In cases where a bulging intervertebral disc contributes to neural impingement, disc material may be removed surgically in a discectomy procedure.

Removal of vertebral bone, as occurs in laminectomy and facetectomy, often leaves the effected area of the spine very unstable, leading to a need for an additional highly invasive fusion procedure that puts extra demands on the patient's vertebrae and limits the patient's ability to move. In a spinal fusion procedure, the vertebrae are attached together with some kind of support mechanism to prevent them from moving relative to one another and to allow adjacent vertebral bones to fuse together. Unfortunately, a surgical spine fusion results in a loss of ability to move the fused section of the back, diminishing the patient's range of motion and causing stress on the discs and facet joints of adjacent vertebral segments. Such stress on adjacent vertebrae often leads to further dysfunction of the spine, back pain, lower leg weakness or pain, and/or other symptoms. Furthermore, using current surgical techniques, gaining sufficient access to the spine to perform a laminectomy, facetectomy and spinal fusion requires dissecting through a wide incision on the back and typically causes extensive muscle damage, leading to significant post-operative pain and lengthy rehabilitation. Discectomy procedures require entering through an incision in the patient's abdomen and navigating through the abdominal anatomy to arrive at the spine. Thus, while laminectomy, facetectomy, discectomy, and spinal fusion frequently improve symptoms of neural and neurovascular impingement in the short term, these procedures are highly invasive, diminish spinal function, drastically disrupt normal anatomy, and increase long-term morbidity above levels seen in untreated patients. Although a number of less invasive techniques and devices for spinal stenosis surgery have been developed, these techniques still typically require removal of significant amounts of vertebral bone and, thus, typically require spinal fusion.

Therefore, it would be desirable to have less invasive methods and devices for cutting, shaving, contouring or otherwise modifying target tissue in a spine to help ameliorate or treat spinal stenosis, while preventing unwanted effects on adjacent or nearby non-target tissues. Ideally, such techniques and devices would reduce neural and/or neurovascular impingement without removing significant amounts of vertebral bone, joint, or other spinal support structures, thereby avoiding the need for spinal fusion and, ideally, reducing the long-term morbidity levels resulting from currently available surgical treatments. It may also be advantageous to have tissue cutting devices capable of treating target tissues in parts of the body other than the spine, while preventing damage of non-target tissues. At least some of these objectives will be met by the present invention.

### SUMMARY OF THE INVENTION

In one aspect of the present invention, a device for cutting ligament and/or bone tissue in a lateral recess and/or an intervertebral foramen of a spine of a patient to treat spinal stenosis may include: an elongate shaft having a rigid proximal portion and a distal portion articulatable relative to the proximal portion; a handle coupled with the proximal portion of the shaft; a tissue cutter disposed on one side of the distal portion of the shaft; a first actuator coupling the handle with the tissue cutter for activating the tissue cutter to cut tissue; and a second actuator coupling the handle with the distal portion for articulating the distal portion relative to the proximal portion. In some embodiments, the distal portion of the shaft may be configured to pass at least partway into an intervertebral foramen of the patient's spine.

By “articulatable,” it is meant that the distal portion may be bent, flexed, angled or the like, relative to the proximal portion. In other words, for the purposes of this application, “articulate” encompasses not only to articulate about a joint, but also includes bending, flexing or angling by means of one or more slits, grooves, hinges, joints or other articulating means.

In various alternative embodiments, the distal portion of the shaft of the device may be rigid, flexible, or part rigid/part flexible. In some embodiments, the distal portion of the shaft may be configured to articulate toward the side on which the tissue cutter is disposed. To make the distal portion of the shaft articulatable relative to the proximal portion, some embodiments may further include an articulation member disposed along the shaft between the proximal and distal portions. As mentioned above, such an articulation member may include, for example, one or more slits, grooves, hinges, joints or the like. In one embodiment, an articulation member may comprise a first material disposed on the side of the shaft on which the tissue cutter is disposed and a second material disposed on an opposite side of the shaft, where the first material is more compressible than the second material.

In some embodiments, the distal portion of the shaft may be configured to articulate incrementally from a relatively unflexed position to a first flexed position and to at least a second flexed position. Optionally, the device may further include a locking mechanism for locking the distal portion in an articulated position relative to the proximal portion.

Any of a number of different tissue cutters may be used in various embodiments. For example, examples of tissue cutters which may be included in the device in some embodiments include but are not limited to blades, abrasive surfaces, files, rasps, saws, planes, electrosurgical devices, bipolar electrodes, monopolar electrodes, thermal electrodes, cold ablation devices, rotary powered mechanical shavers, reciprocating powered mechanical shavers, powered mechanical burrs, lasers, ultrasound devices, cryogenic devices, and water jet devices. In one embodiment, for example, the tissue cutter comprises a translatable blade. In some embodiments, the blade may have a height greater than a height of a portion of the shaft immediately below the blade, and a total height of the blade and the portion of the shaft immediately below the blade may be less than a width of the portion of the shaft immediately below the blade. In some embodiments, the tissue cutter may further include a fixed blade fixedly attached to the shaft, and the translatable blade may move toward the fixed blade to cut tissue. In an alternative embodiment, the tissue cutter may further include a fixed backstop fixedly attached to the shaft, and the translatable blade may move toward the fixed backstop to cut tissue.

In some embodiments, the second actuator may include a tensioning wire extending from the handle to the distal portion of the shaft and a tensioning member on the handle coupled with the tensioning wire and configured to apply tensioning force to the wire. In an alternative embodiment, the second actuator may include a compression member extending from the handle to the distal portion of the shaft and a force application member on the handle coupled with the compression member and configured to apply compressive force to the compression member. In such embodiments, the compression member may include, for example, one or more wires, substrates and/or fluids.

Optionally, in some embodiments the shaft may further include a distal tip articulatable relative to the distal portion of the shaft, and the second actuator may extend to the distal tip. The first and second actuators may have any of a number of different configurations in different embodiments, such as but not limited to triggers, squeezable handles, levers, dials, toggle clamps, toggle switches and/or vice grips.

In another aspect of the present invention, a device for cutting tissue in a human body may include: an elongate shaft having a rigid proximal portion and a distal portion articulatable relative to the proximal portion; a handle coupled with the proximal portion of the shaft; a translatable blade slidably disposed on one side of the distal portion of the shaft; a first actuator coupling the handle with the tissue cutter for activating the tissue cutter to cut tissue; a second actuator coupling the handle with the distal portion for articulating the distal portion relative to the proximal portion; and a locking mechanism configured to lock the distal portion in an articulated configuration relative to the proximal portion. In some embodiments, the translatable blade may have a height greater than a height of a portion of the shaft immediately below the blade, and a total height of the blade and the portion of the shaft immediately below the blade may be less than a width of the portion of the shaft immediately below the blade. In various embodiments, the distal portion of the shaft may be rigid, flexible, or part rigid/part flexible.

In another aspect of the present invention, a method for cutting ligament and/or bone tissue in a lateral recess and/or an intervertebral foramen of a spine of a patient to treat spinal stenosis may involve: advancing a distal portion of a tissue cutting device into an epidural space of the patient's spine; articulating the distal portion relative to a proximal portion of the device; advancing the distal portion at least partway into an intervertebral foramen of the spine; urging a tissue cutter disposed on one side of the distal portion of the device against at least one of ligament or bone tissue in at least one of the lateral recess or the intervertebral foramen; and activating the tissue cutter to cut at least one of the ligament or bone tissue.

In some embodiments, the distal portion may be advanced through an access conduit device. In some embodiments, the distal portion may be advanced through the conduit device and between two adjacent vertebrae into the epidural space without removing vertebral bone. Articulating, in one embodiment, may involve applying tensioning force to a tensioning member disposed longitudinally through the device from the proximal portion to the distal portion. Alternatively, articulating may involve applying compressive force to a compressive member disposed longitudinally through the device from the proximal portion to the distal portion. In some embodiments, articulating may involve articulating to a first articulated configuration before advancing the distal portion into the foramen and further articulating to a second articulated configuration after advancing the distal portion at least partway into the foramen. Some embodiments of the method may optionally further include locking the distal portion in an articulated position relative to the proximal portion before urging the tissue cutter against tissue. Such a method may also involve, in some embodiments, unlocking the distal portion, straightening the distal portion relative to the proximal portion, and removing the tissue cutting device from the patient.

In some embodiments, urging the tissue cutter against tissue may involve applying force to a handle of the tissue cutting device. Activating the tissue cutter, in various embodiments, may involve activating one or more blades, abrasive surfaces, files, rasps, saws, planes, electrosurgical devices, bipolar electrodes, monopolar electrodes, thermal electrodes, cold ablation devices, rotary powered mechanical shavers, reciprocating powered mechanical shavers, powered mechanical burrs, lasers, ultrasound devices, cryogenic devices, and/or water jet devices. For example, in one embodiment, activating the tissue cutter may involve advancing a translatable blade toward one of a stationary blade and a backstop. In an alternative embodiment, activating the tissue cutter may involve retracting a translatable blade toward one of a stationary blade and a backstop. In yet another alternative embodiment, activating the tissue cutter may involve translating two blades toward one another.

These and other aspects and embodiments are described more fully below in the Detailed Description, with reference to the attached Drawings.

### BRIEF DESCRIPTION OF THE DRAWINGS

FIG. 1 is cross-sectional view of a spine, showing a top view of a lumbar vertebra, a cross-sectional view of the cauda equina, and two exiting nerve roots;

FIG. 2 is a left lateral view of the lumbar portion of a spine with sacrum and coccyx;

FIG. 3 is a left lateral view of a portion of the lumbar spine, showing only bone and ligament tissue and partially in cross section;

FIG. 4A is a cross-sectional view of a patient's back and spine with a side view of an articulating rongeur in place for performing a tissue removal procedure, according to one embodiment of the present invention;

FIGS. 4B-4D are side views of the articulating rongeur of FIG. 4A, demonstrating a method for articulating the rongeur and advancing a cutting blade, according to one embodiment of the present invention;

FIGS. 5A and 5B are side cross-sectional views of a distal portion of an articulating rongeur, demonstrating articulation, according to one embodiment of the present invention;

FIGS. 6A and 6B are side cross-sectional views of a distal portion of an articulating rongeur, demonstrating articulation, according to an alternative embodiment of the present invention;

FIG. 7A is a side cross-sectional view of a distal portion of an articulating rongeur, according to an alternative embodiment of the present invention;

FIG. 7B is a magnified side cross-sectional view of a portion of FIG. 7B;

FIG. 7C is an end-on view of the portion of the articulating rongeur of FIG. 7B, from the perspective labeled A in FIG. 7B;

FIG. 8 is a side cross-sectional view of an articulating rongeur, according to an alternative embodiment of the present invention;

FIG. 9 is a side cross-sectional view of an articulating tissue cutting device having a reciprocating file tissue cutter, according to one embodiment of the present invention;

FIG. 10 is a perspective view of an articulating tissue cutting device having a reciprocating file tissue cutter, according to an alternative embodiment of the present invention;

FIG. 11 is a perspective view of an articulating tissue cutting device having a reciprocating file tissue cutter, according to an alternative embodiment of the present invention; and

FIG. 12 a side cross-sectional view of an articulating tissue cutting device having a radiofrequency wire tissue cutter, according to one embodiment of the present invention.

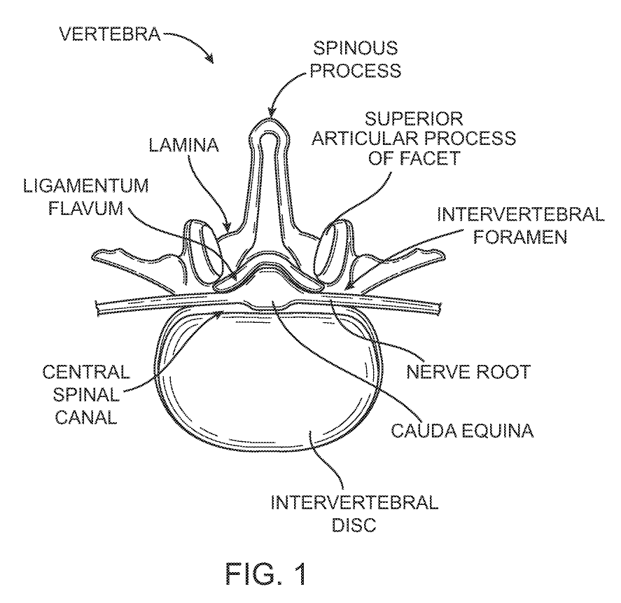

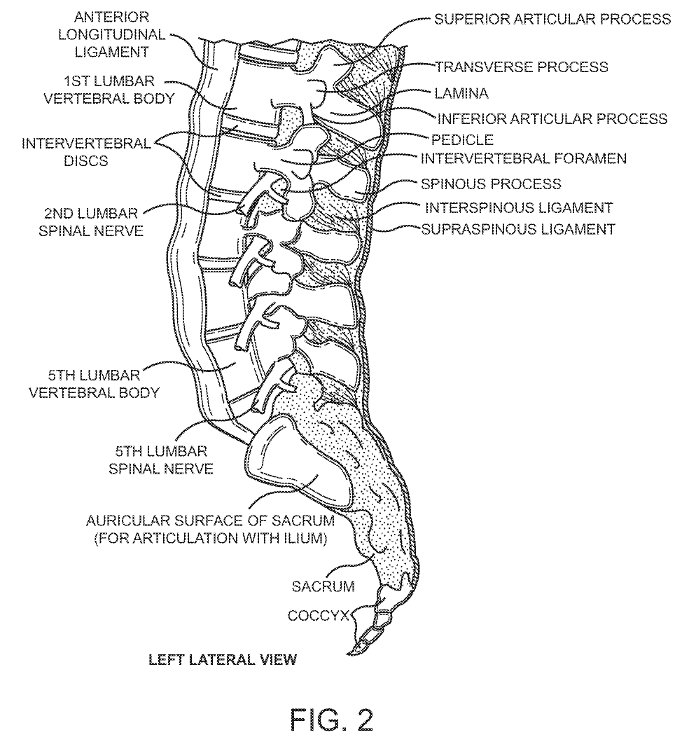

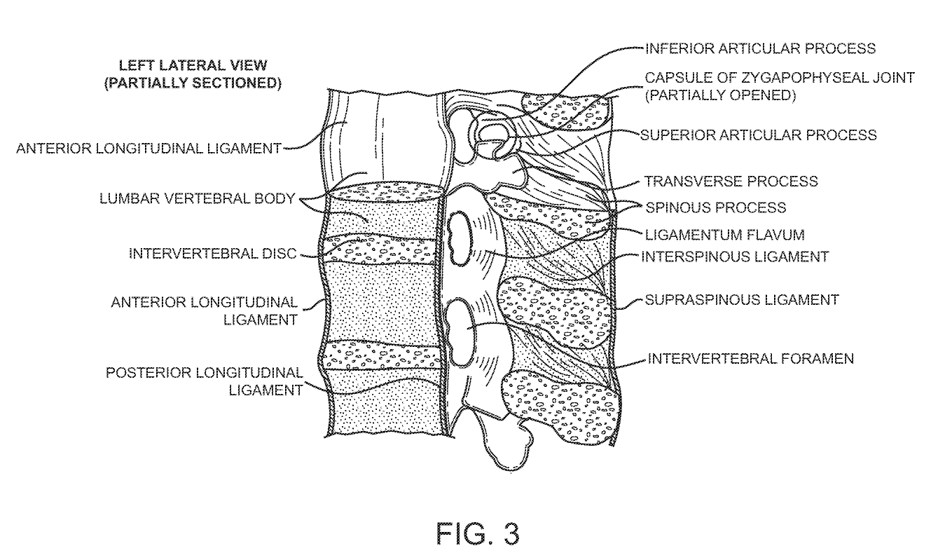

### DETAILED DESCRIPTION OF THE INVENTION

Various embodiments of an articulating tissue cutting device for modifying tissue in a patient are provided. Although portions of the following description and accompanying drawing figures generally focus on cutting tissue in a spine, in various embodiments, any of a number of tissues in other anatomical locations in a patient may be modified.

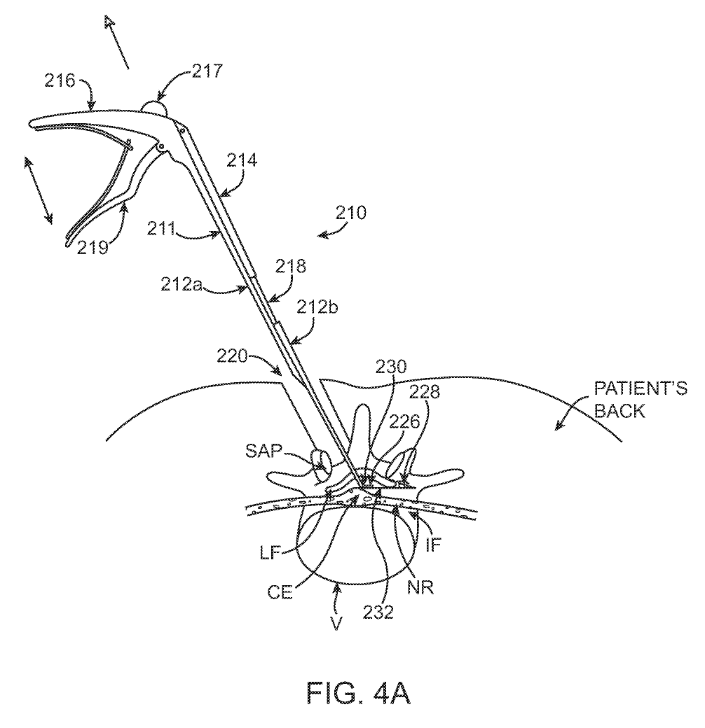

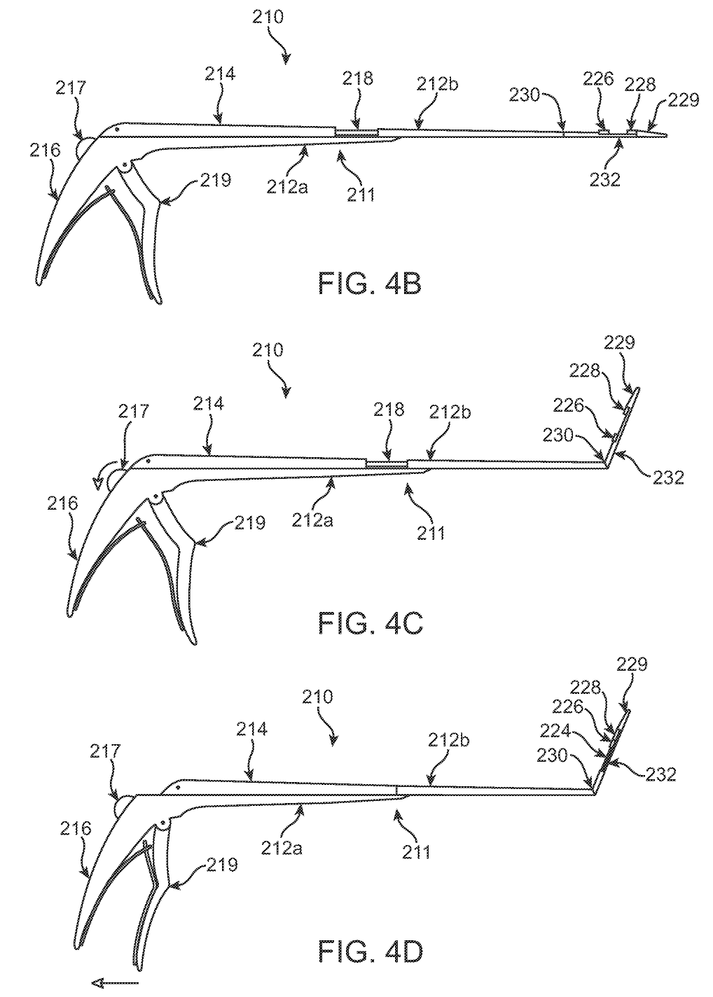

Referring to FIG. 4A, one embodiment of articulating rongeur 210 may include a shaft having a proximal portion 211, a distal portion 232, and an articulation feature 230 (or “articulation member”) between the two. A handle 216 with a squeezable trigger 219 and a dial 217 may be coupled with proximal shaft portion 211. A proximal blade 226 and a distal blade 228 may be disposed along distal shaft portion 232. In some embodiments, both proximal shaft portion 211 and distal shaft portion 232 are predominantly rigid. In alternative embodiments, distal shaft portion 232 may be more flexible than proximal portion 211 or may be largely rigid but may have one or more flexible portions disposed along its length. Proximal shaft portion 211 may include a proximal stationary portion 212a coupled with or extending from proximal handle 216, a distal stationary portion 212b, and a movable shaft portion 214. Articulation feature 230 may include any suitable mechanism, such as one or more slits, grooves, hinges, joints and/or combinations of materials, to allow distal portion 232 to articulate relative to proximal portion 211. As mentioned above, “articulate” includes articulating about a joint, as well as bending, flexing, angling and the like. Distal shaft portion 232 may include a portion that extends underneath and between blades 226, 228, which may be referred to as a “substrate,” “platform” or “extension” herein.

In one embodiment, at least two flexible wires 224 (or “wire bundle”—see FIG. 4D) may slidably extend through a portion of proximal shaft portion 211 and distal shaft portion 232 so that their distal ends attach to proximal blade 226. Optionally, wires 224 may be bundled together along their entire lengths or along part of their lengths, and such a wire bundle may be partially housed within a wire bundle tube 218, which may slidably pass through distal stationary shaft portion 212b. In use, trigger 219 may be squeezed (double-headed, solid-tipped arrow) to advance moveable shaft portion 214, which advances wire bundle tube 218 and wires 224, thus advancing proximal blade 226 toward stationary blade 228 to cut tissue.

In some embodiments, articulating rongeur 210 may be advanced into a patient's back through an incision 220, which is shown in FIG. 4A as an open incision but which may be a minimally invasive or less invasive incision in alternative embodiments. Rongeur 210 may be advanced into the patient in a relatively straight configuration and then articulate (or “flexed” or “bent”) at articulation feature 230 to facilitate passing at least part of distal shaft portion 232 into an intervertebral foramen (IF). In some embodiments, an articulating member on handle 216, such as dial 217, may be used to apply a force to a flexing member extending from dial 217 to at least articulation feature 230. The ability of rongeur 210 to articulate about articulation feature 230 may facilitate passage of rongeur 210 between tissues in hard-to-reach or tortuous areas of the body, such as between a nerve root (NR) and facet joint and into an intervertebral foramen (IF). Generally, rongeur 210 may be advanced to a position such that blades 226, 228 face tissue to be cut in a tissue removal procedure (“target tissue”) and one or more non-cutting surfaces of rongeur 210 face non-target tissue, such as nerve and/or neurovascular tissue. In the embodiment shown in FIG. 4A, blades 226, 228 are positioned to cut ligamentum flavum (LF) and may also cut hypertrophied bone of the facet joint, such as the superior articular process (SAP). (Other anatomical structures depicted in FIG. 4A include the vertebra (V) and cauda equina (CE)).

Once rongeur 210 is advanced into the patient to position distal portion 232 at least partway into an intervertebral foramen, articulation feature 230 may be locked into position, either by a locking mechanism in articulation feature 230 itself or alternatively or additionally by a locking mechanism in handle 216, such as a mechanism coupled with or part of dial 217. Once articulation feature 230 is locked, handle 16 may be pulled (hollow-tipped arrow) to pull distal shaft portion 232 against target tissue and thus to urge the cutting portion of rongeur 210 (e.g., blades 226, 228) against ligamentum flavum (LF), superior articular process (SAP), and/or other target tissue to be cut. Handle 216 may then be actuated, such as by squeezing in the embodiment shown, which advances moveable shaft 214, thus advancing wire bundle tube 218, flexible wires 224 and proximal blade 226, to cut tissue between proximal blade 226 and distal blade 228. Handle 216 may be released and squeezed as many times as desired to remove a desired amount of tissue. When a desired amount of tissue has been cut (or at any point during a tissue cutting procedure to monitor progress), rongeur 210 may be removed from the patient's back.

As mentioned previously, and as described in greater detail below, in various embodiment articulation feature 230 may take any of a number of different forms and may generally include any suitable feature or features to allow rongeur 210 to flex or be flexed. In various embodiments, articulation feature 230 may include one or more hinges, slits, grooves, joints, materials having varying levels of compressibility or the like.

Referring now to FIGS. 4B-4D, the articulating and blade advancing functions of articulating rongeur 210 are demonstrated. FIG. 4B shows articulating rongeur 210 in its generally straight configuration. In one embodiment, as shown in FIG. 4C, dial 217 may be turned (hollow-tipped arrow) to articulate distal portion 232. With distal portion 232 articulated, as shown in FIG. 4D, trigger 219 may be squeezed (hollow-tipped arrow) to advance moveable shaft portion 214, which in turn advances wires 224 and proximal blade 226 toward distal blade 228 to cut target tissue. In some embodiments, proximal blade 226 may be advanced while rongeur is in its straight or articulated configuration. In some embodiments, rongeur 210 may articulate in increments, such as from a straight configuration to a first flexed configuration to a second flexed configuration and so on. Also in some embodiments, articulation feature 230 may automatically lock into an articulated position. In alternative embodiments, articulation feature 230 may be manually locked, such as by locking dial 217 or the like.

For further detail regarding a multi-wire tissue cutter device, many of the features of which may be incorporated into articulating rongeur 210, reference may be made to U.S. patent application Ser. No. 11/______ (Attorney Docket No. 026445-000910US), titled “Multi-Wire Tissue Cutter,” and filed on Aug. 1, 2006, the full disclosure of which is hereby incorporated by reference. In alternative embodiments, different tissue cutting mechanisms may be included in articulating rongeur 210. For example, in one embodiment, distal blade 228 may be translatable and proximal blade 226 may be stationary. In an alternative embodiment, distal blade 228 and proximal blade 226 may be translated toward one another to cut tissue. A number of such bladed tissue cutting mechanisms are described, for example, in U.S. patent application Ser. No. 11/405,848 (Original Attorney Docket No. 78117-200301), titled “Mechanical Tissue Modification Devices and Methods,” and filed on Apr. 17, 2006, the full disclosure of which is hereby incorporated by reference. In further alternative embodiments, some of which are described in greater detail below, blades 226, 228 may be replaced altogether by a different tissue cutting mechanism, such as but not limited to one or more abrasive surfaces, files, rasps, saws, planes, electrosurgical devices, bipolar electrodes, monopolar electrodes, thermal electrodes, cold ablation devices, rotary powered mechanical shavers, reciprocating powered mechanical shavers, powered mechanical burrs, lasers, ultrasound devices, cryogenic devices, and/or water jet devices

Generally, proximal shaft portion 211 and distal shaft portion 232 may be formed of any suitable material, such as but not limited to stainless steel. Wire bundle 224 extends through at least part of wire tube 218, through distal stationary shaft portion 212b, and in some embodiments through part of distal shaft portion 232, and is coupled with proximal blade 226. Wire tube 218 acts to secure the proximal end of wire bundle 224, such as by crimping, welding or the like. In alternative embodiments, wire tube 218 may be excluded, and the proximal end of wire bundle 224 may be otherwise coupled with device. For example, in various embodiments, wire bundle 224 may be coupled with moveable shaft portion 214, may be movably coupled with handle 216, or the like. In the side view of FIG. 4D, wire bundle 224 appears as a single wire, in this embodiment due to the fact that distal shaft portion 232 flattens wire bundle 224 to a one-wire-thick cross section.

In various embodiments, proximal shaft portion 211 and distal shaft portion 232 may have any suitable shapes and dimensions and may be made of any suitable materials. For example, in various embodiments, shaft portions 211, 232 may be made from any of a number of metals, polymers, ceramics, or composites thereof. Suitable metals, for example, may include but are not limited to stainless steel (303, 304, 316, 316L), nickel-titanium alloy, tungsten carbide alloy, or cobalt-chromium alloy, for example, Elgiloy® (Elgin Specialty Metals, Elgin, Ill., USA), Conichrome® (Carpenter Technology, Reading, Pa., USA), or Phynox® (Imphy SA, Paris, France). Suitable polymers include but are not limited to nylon, polyester, Dacron®, polyethylene, acetal, Delrin® (DuPont, Wilmington, Del.), polycarbonate, nylon, polyetheretherketone (PEEK), and polyetherketoneketone (PEKK). In some embodiments, polymers may be glass-filled to add strength and stiffness. Ceramics may include but are not limited to aluminas, zirconias, and carbides.

Portions of shaft 211, 232 through which wire bundle 224 travels will generally be predominantly hollow, while other portions may be either hollow or solid. For example, in one embodiment, moveable shaft portion 214 and proximal stationary portion 212a may be solid, and distal stationary portion 212b and part of distal portion 232 may be hollow. Although one particular embodiment of a shaft mechanism for moving wire bundle 224 is shown, various embodiments may employ any of a number of alternative mechanisms.

Wire bundle 224 may include as few as two flexible wires 224 and as many as one hundred or more wires 224. In some embodiments, for example, between three and 20 wires 224 may be used, and even more preferably, between four and ten wires 224. Wires 224 may have any of a number of different diameters, so in some embodiments the number of wires 224 used may be determined by the diameter of wire 224 used. In various embodiments, each wire 224 may be a solid wire, a braided wire, a core with an outer covering or the like, and may be made of any suitable material. For example, in various embodiments, wires 224 may be made from any of a number of metals, polymers, ceramics, or composites thereof. Suitable metals, for example, may include but are not limited to stainless steel (303, 304, 316, 316L), nickel-titanium alloy, tungsten carbide alloy, or cobalt-chromium alloy, for example, Elgiloy® (Elgin Specialty Metals, Elgin, Ill., USA), Conichrome® (Carpenter Technology, Reading, Pa., USA), or Phynox® (Imphy SA, Paris, France). In some embodiments, materials for the wires 224 or for portions or coatings of the wires may be chosen for their electrically conductive or thermally resistive properties. Suitable polymers include but are not limited to nylon, polyester, Dacron®, polyethylene, acetal, Delrin® (DuPont, Wilmington, Del.), polycarbonate, nylon, polyetheretherketone (PEEK), and polyetherketoneketone (PEKK). In some embodiments, polymers may be glass-filled to add strength and stiffness. Ceramics may include but are not limited to aluminas, zirconias, and carbides. In some embodiments, all wires 224 may be made of the same material, whereas in alternative embodiments, wires 224 may be made of different materials. Individual wires 224 may also have any length, diameter, tensile strength or combination of other characteristics and features, according to various embodiments, some of which are discussed in greater detail below.

In various embodiments, flexible wires 224 may be bound or otherwise coupled together at one or more coupling points or along the entire length of wire bundle 224. In one embodiment, for example, wires 224 may be coupled together by a sleeve or coating overlaying wire bundle 224. In another embodiment, wires 224 may only be coupled together at or near their proximal ends, at or near their connection point to tube 218, moveable shaft portion 214 or the like. In an alternative embodiment, wires 224 may be individually coupled with an actuator, such as handle 216, and not coupled to one another directly. In any case, wires 224 will typically be able to move at least somewhat, such as laterally, relative to one another.

In some embodiments, wire bundle 224 may include one or more elongate, flexible members for performing various functions, such as enhancing tissue cutting, visualizing a target area or the like. For example, in various embodiments, wire bundle 224 may include one or more optical fibers, flexible irrigation/suction tubes, flexible high pressure tubes, flexible insulated tubing for carrying high temperature liquids, flexible insulated tubing for carrying low temperature liquids, flexible elements for transmission of thermal energy, flexible insulated wires for the transmission of electrical signals from a sensor, flexible insulated wires for the transmission of electrical signals towards the distal end of the wires, energy transmission wires, or some combination thereof. Examples of visualization devices that may be used include flexible fiber optic scopes, CCD (charge-coupled device) or CMOS (complementary metal-oxide semiconductor) chips at the distal end of flexible probes, LED illumination, fibers or transmission of an external light source for illumination or the like.

When blades 226, 228 face target tissue to be modified, such as buckled, thickened or otherwise impinging ligamentum flavum tissue, rongeur 210 is configured such that an atraumatic surface (or multiple atraumatic surfaces) of the distal shaft portion 232 faces non-target tissue. Distal shaft portion 232 may thus act as a tissue protective surface and in various embodiments may have one or more protective features, such as a width greater than the width of blades 226, 228, rounded edges, bumpers made of a different material such as a polymer, protective or lubricious coating(s), extendable or expandable barrier member(s), drug-eluting coating or ports, or the like. In some instances, distal shaft portion 232 may include one or more “non-tissue-modifying” surfaces, meaning that such surfaces may not substantially modify the non-target tissue. In alternative embodiments, distal shaft portion 232 may affect non-target tissue by protecting it in some active way, such as by administering one or more protective drugs, applying one or more forms of energy, providing a physical barrier, or the like.

Generally, blades 226, 228 may be disposed on distal shaft portion 232. Proximal blade 226 may be unattached or moveably/slidably attached to distal shaft portion 232, so that it is free to translate (or “reciprocate”) along distal shaft portion 232 with the back and forth movement of wire bundle 224. In one embodiment, for example, proximal blade 226 may be slidably coupled with distal shaft portion 232 via a piece of material wrapped around blade 226 and distal shaft portion 232. In another embodiment, proximal blade 226 may slide through one or more tracks on distal shaft portion 232. Distal blade 228 may be fixedly attached to distal shaft portion 232 and thus remain stationary, relative to distal shaft portion 232, such that proximal blade 226 translates toward stationary distal blade 228 to cut tissue. In alternative embodiments, the distal end of wire bundle 224, itself, may be used to cut tissue, and rongeur 210 may thus not include proximal blade 226. For example, each wire 224 may have a sharp, tissue cutting point, or wire bundle 224 as a whole may form a sharp, tissue cutting edge. The distal end of wire bundle 224 may advance toward distal blade 228 to cut target tissue, or in alternative embodiments, wire bundle 224 may advance toward a non-sharp backstop to cut tissue or may simply advance against tissue to ablate it, without pinching the tissue between the wire bundle 224 distal end and any other structure. An example of the latter of these embodiments might be where ultrasound energy is used to reciprocate wire bundle 224, in which case the reciprocation of wire bundle 224 may be sufficient to cut or ablate tissue, without pinching or snipping between wire bundle and another structure.

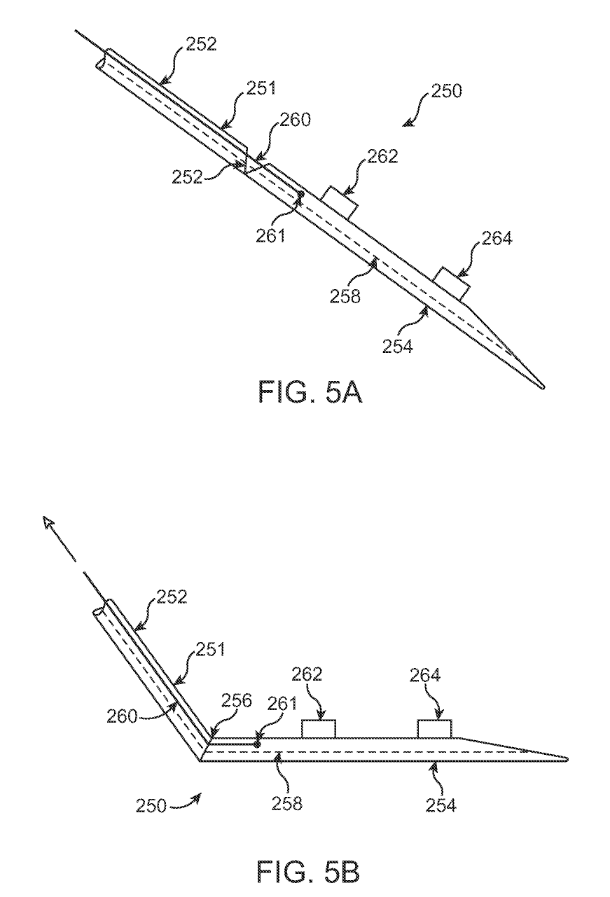

In various embodiments, blades 226, 228, or other cutting structures such as the distal ends of wire bundle 224, a backstop or the like, may be disposed along any suitable length of distal shaft portion 232. In the embodiment shown in FIG. 5A, for example, blades 226, 228 are disposed along a length of distal shaft portion 232. In an alternative embodiment, distal shaft portion 232 may comprise a hollow portion through which wire bundle 224 travels and a window through which wire bundle 224 is exposed. In any case, blades 226, 228 or other cutting members may be disposed or exposed along a desired length of rongeur 210, to help limit an area in which the cutting members are active, thus helping to limit the exposure of non-target tissues to such cutting elements. In one embodiment, for example, such as an embodiment of the device to be used in a spinal treatment, blades 226, 228 may be disposed along a length of distal shaft portion 232 measuring no longer than about 10 cm, and preferably no more than about 6 cm, and even more preferably no more than about 3 cm. In various embodiments, the length along which blades 226, 228 are disposed may be selected to approximate a length of a specific anatomical treatment area.

000531 Blades 226, 228 may be made from any suitable metal, polymer, ceramic, or combination thereof. Suitable metals, for example, may include but are not limited to stainless steel (303, 304, 316, 316L), nickel-titanium alloy, tungsten carbide alloy, or cobalt-chromium alloy, for example, Elgiloy® (Elgin Specialty Metals, Elgin, Ill., USA), Conichrome® (Carpenter Technology, Reading, Pa., USA), or Phynox® (Imphy SA, Paris, France). In some embodiments, materials for blades 226, 228 or for portions or coatings of blades 226, 228 may be chosen for their electrically conductive or thermally resistive properties. Suitable polymers include but are not limited to nylon, polyester, Dacron®, polyethylene, acetal, Delrin® (DuPont, Wilmington, Del.), polycarbonate, nylon, polyetheretherketone (PEEK), and polyetherketoneketone (PEKK). In some embodiments, polymers may be glass-filled to add strength and stiffness. Ceramics may include but are not limited to aluminas, zirconias, and carbides. In various embodiments, blades 226, 228 may be manufactured using metal injection molding (MIM), CNC machining, injection molding, grinding and/or the like. Proximal and distal blades 226, 228 may be attached to wire bundle 224 and distal shaft portion 232, respectively, via any suitable technique, such as by welding, adhesive or the like.

In some embodiments, articulating rongeur 210 may include a tissue collection chamber 229 distal to distal blade 228. For example, distal blade 228 may be hollow and in fluid communication with tissue collection chamber 229, such that when tissue is cut using blades, 226, 228, at least some of the tissue passes under distal blade 228 and into collection chamber 229. Tissue collection chamber 229 may be made of any suitable material, such as but not limited to any of the materials listed above for making blades 226, 228. In one embodiment, for example, chamber 229 may comprise a layer of polymeric material attached between distal blade 228 and distal shaft portion 232. In another embodiment, collection chamber 229 and distal blade 228 may comprise one continuous piece of material, such as stainless steel. Generally, distal blade 228 and chamber 229 form a hollow, continuous space into which at least a portion of cut tissue may pass after it is cut.

With reference now to FIGS. 5A and 5B, a portion of an articulating rongeur 250, according to one embodiment, may include a shaft 251 having a longitudinal axis 258, a proximal shaft portion 252, a distal shaft portion 254, and an articulation feature 256 between the proximal and distal portions 252, 254. Rongeur 250 may also include a proximal blade 262 and a distal blade 264 disposed on the distal shaft portion 254. (In FIGS. 5A and 5B, mechanism for moving one or both of blades 262, 264 is omitted, to enhance the clarity of the drawing figures.) Rongeur 250 may further include one or more tensioning wires 260, extending from a handle at the proximal end of rongeur 250 (not shown), through proximal shaft portion 252, to an attachment point 261 in or on distal shaft portion 254.

Tensioning wire 260 generally extends through and is attached to shaft 251 closer to the top/blade side than the bottom/opposite side, relative to longitudinal axis 258. When tensioning wire 260 is pulled proximally, as depicted by the hollow-tipped arrow in FIG. 5B, shaft 251 articulates, bends or flexes toward the blade side of shaft 251 by articulating at articulation feature 256. In various embodiments, articulation feature 256 may include any suitable number of slits, grooves, hinges, joints or the like. In one embodiment, for example, articulation feature 256 may include two materials on opposite sides of shaft 251, with a more easily compressible material located on the top side (or blade side) of articulation feature 256 and a less easily compressible material located on the opposite/bottom side.

In some embodiments, tensioning wire 260 may extend only to a distal side of articulation feature 256 and attach there, rather than extending into distal shaft portion 254. Alternatively, tensioning wire 260 may extend farther distally on distal portion 254, to attach at a point at or near distal blade 264 or even at or near the extreme distal end of shaft 251. In such cases, a sufficient amount of tensioning force applied to tensioning wire 260 may cause distal portion 254 to curl or bend in the direction of the blade side of shaft 251. If distal portion 254 is made of a relatively rigid material, such bending may be minimal, while if distal portion 254 is made of a more flexible material, such bending may be more significant. In some cases, such bending may facilitate passage of distal portion 254 around a curved surface, through an anatomical curved passage between tissues, or the like. For example, in some embodiments, distal shaft portion 254 may be made of a relatively flexible material, which may facilitate its passage into a small space, between tissues or the like. Applying tensioning force via tensioning wire 260 may, in such an embodiment, not only articulate shaft 251 at articulation feature 256, but may also stiffen or rigidify distal portion 254, so that device 250 may be pulled back to urge the stiffened/rigidified distal portion 254 against target tissue.

Tensioning wire 260 generally comprises a high-strength wire, cable, cord or the like and may be made of any suitable material. In one embodiment, for example, tensioning wire 260 may be made of carbon fiber. Other suitable metals from which tensioning wires 260 may be constructed may include but are not limited to stainless steel (303, 304, 316, 316L), nickel-titanium alloy, tungsten carbide alloy, or cobalt-chromium alloy, for example, Elgiloy® (Elgin Specialty Metals, Elgin, Ill., USA), Conichrome® (Carpenter Technology, Reading, Pa., USA), or Phynox® (Imphy SA, Paris, FranceSuitable polymers include but are not limited to nylon, polyester, Dacron®, polyethylene, acetal, Delrin® (DuPont, Wilmington, Del.), polycarbonate, nylon, polyetheretherketone (PEEK), and polyetherketoneketone (PEKK). In some embodiments, polymers may be glass-filled to add strength and stiffness. Ceramics may include but are not limited to aluminas, zirconias, and carbides.

In various embodiments, any number of tensioning wires 260 may be used, such as between one and 100 wires 260. In cases where multiple wires 260 are used, it may be possible in some embodiments to further steer distal shaft portion 254 by individually manipulating one or more wires 260 relative to other wires. In one embodiment, tensioning wires 260 may extend through a lumen of shaft 251 and may be attached at attachment point 261 via any suitable means, such as adhesive, welding, crimping, pressure fitting or the like. In some embodiments, tensioning wire 260 may be sufficiently strong that an amount of tensioning force may be applied that can bend distal portion 254 and/or render distal portion 254 more stiff or rigid.

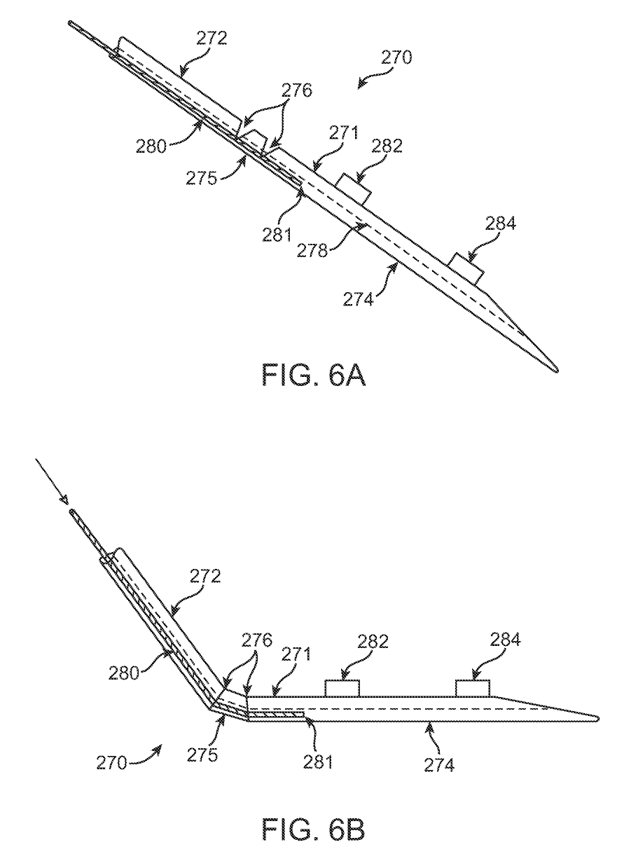

In an alternative embodiment, and with reference now to FIGS. 6A and 6B, a portion of an articulating rongeur 270 may include a shaft 271 having a longitudinal axis 278, a proximal shaft portion 272, a distal shaft portion 274, and an articulation feature 275 including multiple flex slits 276. Rongeur 270 may also include a proximal blade 282 and a distal blade 284 disposed on the distal shaft portion 274. (Again, in FIGS. 6A and 6B, mechanism for moving one or both of blades 282, 284 is omitted, to enhance the clarity of the drawing figures.) Rongeur 270 may further include one or more compression members 280, extending from a handle at the proximal end of rongeur 270 (not shown), through proximal shaft portion 272, to at least articulation feature 275, and in some embodiments (as in FIGS. 6A and 6B) to an attachment point 281 in distal shaft portion 274.

As described above, in various embodiments, articulation feature 275 may include any suitable number of flex slits 276, grooves, hinges, joints, differing materials or the like. Compression member 280 extends through shaft 271 closer to the bottom/opposite side than the top/blade side, relative to longitudinal axis 278. When compressive (or “pushing”) force is applied to compression member 280, as depicted by the hollow-tipped arrow in FIG. 6B, shaft 271 bends or flexes toward the blade side of shaft 271 by bending/flexing at articulation feature 275.

In some embodiments, compression member 280 may extend only to a distal side of articulation feature 275 and attach there, rather than extending into distal shaft portion 274. Alternatively, compression member 280 may extend farther distally on distal portion 274, to attach at a point at or near distal blade 284 or even at or near the extreme distal end of shaft 271. In such cases, a sufficient amount of compressive force applied to compression member 280 may cause distal portion 274 to curl or bend in the direction of the blade side of shaft 271. If distal portion 274 is made of a relatively rigid material, such bending may be minimal, while if distal portion 274 is made of a more flexible material, such bending may be more significant. In some cases, such bending may facilitate passage of distal portion 274 around a curved surface, through an anatomical curved passage between tissues, or the like. For example, in some embodiments, distal shaft portion 274 may be made of a relatively flexible material, which may facilitate its passage into a small space, between tissues or the like. Applying tensioning force via compression member 280 may, in such an embodiment, not only articulate shaft 271 at articulation feature 275, but may also stiffen or rigidify distal portion 274, so that device 270 may be pulled back to urge the stiffened/rigidified distal portion 274 against target tissue.

Compression member 280 may generally comprise any of a number of force transmitting members, such as one or more high-strength wires, a material substrate, a column of fluid or the like. A wire, substrate or other solid compression member 280 may be made of any suitable material, such as but not limited to carbon fiber, stainless steel (303, 304, 316, 316L), nickel-titanium alloy, tungsten carbide alloy, or cobalt-chromium alloy, for example, Elgiloy® (Elgin Specialty Metals, Elgin, Ill., USA), Conichrome® (Carpenter Technology, Reading, Pa., USA), or Phynox® (Imphy SA, Paris, FranceSuitable polymers include but are not limited to nylon, polyester, Dacron®, polyethylene, acetal, Delrin® (DuPont, Wilmington, Del.), polycarbonate, nylon, polyetheretherketone (PEEK), and polyetherketoneketone (PEKK). In some embodiments, polymers may be glass-filled to add strength and stiffness. Ceramics may include but are not limited to aluminas, zirconias, and carbides.

In various embodiments, any number of compression members 280 may be used, such as between one and 100 compression wires or the like. In cases where multiple compression members 280 are used, it may be possible in some embodiments to further steer distal shaft portion 274 by individually manipulating one or more compression members 280 relative to others. In one embodiment, compression member 280 may extend through a lumen of shaft 271 and may be attached at attachment point 281 via any suitable means, such as adhesive, welding, crimping, pressure fitting or the like. In one embodiment, for example, compression member 280 may abut a structure such as a backstop, screw drive or the like. In some embodiments, compression member 280 may be sufficiently strong that an amount of tensioning force may be applied that can bend distal portion 274 and/or render distal portion 274 more stiff or rigid.

In one alternative embodiment (not shown), a rongeur may include both one or more tensioning members 260 and one or more compression members 280. In such an embodiment, both tensioning and compression force may be applied to the rongeur to flex its shaft at one or more locations along its length.

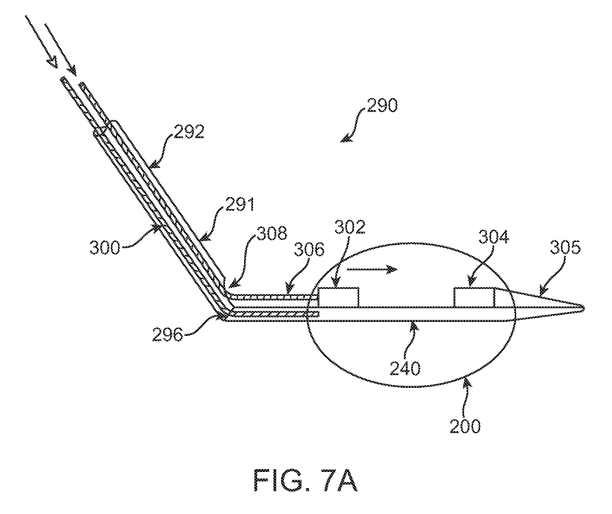

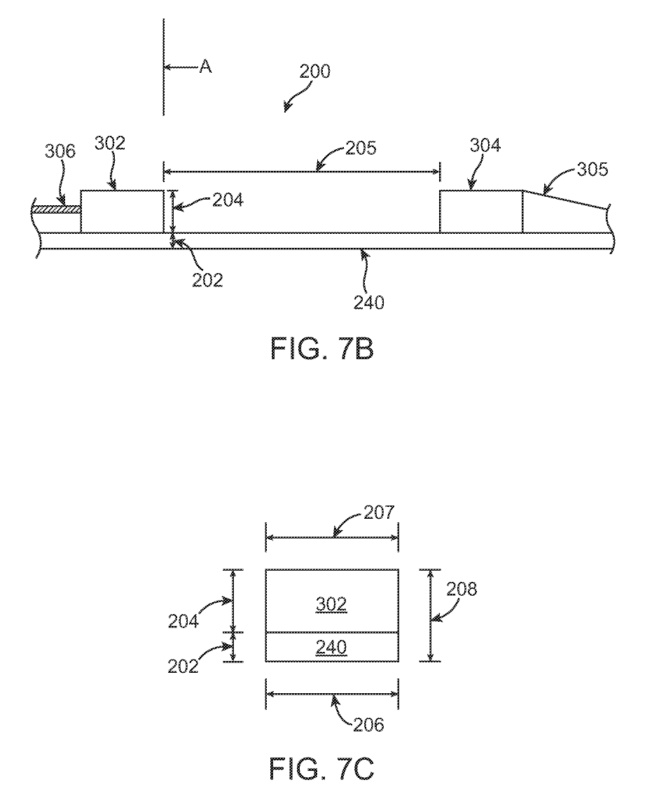

Referring now to FIG. 7A, another embodiment of an articulating rongeur 290 is shown in cross-section. Articulating rongeur 290 (of which only a portion is shown) may include a shaft 291 having a proximal shaft portion 292, a distal shaft platform 240 (or “substrate” or “extension”), and an articulation feature 296. Rongeur 290 may also include a proximal blade 302, slidably disposed on platform 240 and coupled with a blade actuating wire 306 that extends through proximal shaft portion 292 and out an aperture 308 therein. A distal blade 304 may be fixedly attached to platform 240, and a tissue capture member 305 may be disposed between distal blade 304 and platform 240 to capture cut tissue that passes under blade 304. Rongeur 290 may further include one or more compression members 300, as described above in reference to FIGS. 6A and 6B. Compressive force may be applied to compression member 300 (hollow-tipped arrow) to articulate rongeur 290 about articulation feature 296, and blade articulating wire 306 may be advanced to advance proximal blade 302 (solid-tipped arrows) to cut tissue.

In various embodiments, platform 240 may comprise an extension of a lower surface of proximal shaft portion 292. Alternatively or additionally, platform 240 may comprise one or more separate pieces of material coupled with proximal shaft portion 292, such as by welding or attaching with adhesive. Platform 240 may comprise the same or different material(s) as proximal shaft portion 292, according to various embodiments, and may have any of a number of configurations. For example, platform 240 may comprise a flat, thin, flexible strip of material (such as stainless steel). In an alternative embodiment, platform 240 may have edges that are rounded up to form a track through which proximal blade 302 may travel. In some embodiments, platform 240 may be flexible, allowing it to bend, while in other embodiments, platform 240 may be predominantly rigid, so that it does not bend or bends only slightly when compressive force is applied to compressive member 300. In various embodiments, platform 240 may be made more rigid by making platform 240 more think and/or by using more rigid material to construct platform 240. In some embodiments, platform 240 may be made of a shape memory material and given a curved shape, while in other embodiments platform 240 may be rigid and curved or rigid and straight. Differently shaped platforms 240 and/or platforms 240 having different amounts of flexibility may facilitate use of different embodiments of rongeur 290 in different locations of the body. A more rigid platform 240, for example, may facilitate cutting of a hard material such as bone with blades 302, 304.

Some embodiments of rongeur 290 may further include one or more electrodes coupled with platform 240, for transmitting energy to tissues and thereby confirm placement of rongeur 290 between target and non-target tissues. For example, one or more electrodes may be placed on a lower surface of platform 240, and the electrode(s) may be stimulated to help confirm the location of neural tissue relative to blades 302, 304. In such embodiments, nerve stimulation may be observed as visible and/or tactile muscle twitch and/or by electromyography (EMG) monitoring or other nerve activity monitoring. In various alternative embodiments, additional or alternative devices for helping position, use or assess the effect of rongeur 210 may be included. Examples of other such devices may include one or more neural stimulation electrodes with EMG or SSEP monitoring, ultrasound imaging transducers external or internal to the patient, a computed tomography (CT) scanner, a magnetic resonance imaging (MRI) scanner, a reflectance spectrophotometry device, and a tissue impedance monitor disposed across a bipolar electrode tissue modification member or disposed elsewhere on rongeur 210.

Referring now to FIGS. 7B and 7C, a side view (FIG. 7B) and an end-on view (FIG. 7C) of a portion 200 of rongeur 290 (circled in FIG. 7A) are shown. (FIG. 7C is a view from the perspective labeled A in FIG. 7B.) It has been found that in some embodiments, various components and portions of tissue cutting rongeur 290 may preferably have a combination of dimensions that facilitate passage into a small space and effective tissue cutting. In various embodiments, the dimensions described below may be applied to any tissue cutting device, especially devices designed to cut tissue located in small anatomical passageways or spaces, such as in and around an intervertebral foramen of a spine. For example, a number of alternative tissue cutting devices are described in U.S. patent application Ser. No. 11/405,848, entitled “Mechanical Tissue Modification Devices and Methods” (Original Attorney Docket No. 78117-200301), and filed Apr. 17, 2006, the full disclosure of which is hereby incorporated by reference. In that disclosure, for example, one of the embodiments a tissue cutting device includes a translatable blade that is retracted via two pull wires. It is contemplated that the dimensional characteristics described below may be applied to such a device, as well as to other tissue cutting devices in other alternative embodiments.

Referring again to FIGS. 7B and 7C, in one embodiment, platform 240 (or “substrate”) may have a substrate height 202 (or “thickness”), blades 302, 304 may have a blade height 204, edges of blades 302, 304 may be separated by a blade opening distance 205, blades 302, 304 may have a blade width 207, platform 240 may have a substrate width 206, and each blade 26, 28 together with platform 240 may have a total device height 208. (Substrate height 202 or substrate width 206 may also be referred to as the height or width of “a portion of the shaft immediately below the blade(s).”) Each of these various dimensions may be adjusted according to various embodiments and for various applications to different parts of patient anatomy. Some embodiments, for example, may be configured for use in and near an intervertebral foramen of a spine. In an alternative embodiment, dimensions of rongeur 290 may be selected for use in a shoulder surgery procedure, a knee surgery procedure, a hand surgery procedure or the like.

In some embodiments, the portion 200 of rongeur 290 may have an overall size and dimensions such that it may be passed into an epidural space of a spine and at least partially into an intervertebral space of the spine, so that it may be used to cut ligament and/or bone in the spine to treat neural and/or neurovascular impingement. In some embodiments, for example, substrate height 202 may be less than blade height 204. In other words, the ratio of substrate height 202 to blade height may be approximately less than one, and in some embodiments approximately less than or equal to ¾. In these or other embodiments, total height 208 (of blade 302 and platform 240) may be less than substrate width 206 and/or blade width 207. (In some embodiments, substrate width 206 may be approximately equal to blade width 207, as shown, while in alternative embodiments, substrate width 206 may be greater than blade width 207.) In other words, the ratio of total height 208 to width 207 may be approximately less than one, and in some embodiments approximately less than or equal to ¾. In some embodiments, rongeur 290 may have a combination of a ratio of substrate height 202 to blade height approximately less than one and a ratio of total height 208 to width 206 approximately less than one. Such a configuration is contrary to that of traditional rongeurs, which include cutting blades thinner than their underlying supporting structure and which have a total height greater than the width of the device. In one embodiment, for example, blade opening distance 205 may be between about 0.1 inches and about 0.5 inches, substrate height 202 may be between about 0.010 inches and about 0.050 inches, blade height 204 may be between about 0.010 inches and about 0.075 inches, and blade width 207 may be between about 0.2320 and about 0.400 inches. More preferably, in one embodiment, blade opening distance 205 may be between about 0.3 inches and about 0.35 inches, substrate height 202 may be between about 0.025 inches and about 0.035 inches, blade height 204 may be between about 0.040 inches and about 0.060 inches, and blade width 207 may be between about 0.165 and about 0.250 inches. In alternative embodiments, such as for use in other parts of the body, rongeur 290 may have any of a number of different combinations of dimensions.

To optimize rongeur 290 for any of a number of possible uses, the dimensions described above may be combined with any of a number of materials for the various components of rongeur 290. Examples of such materials for blades 302, 304, platform 240 and the like have been listed previously. In some embodiments, for example, platform 240 may be made of a material and may have a height or thickness 202 such that it is predominantly stiff or rigid, even when placed under tension against a rounded surface. In another embodiment, platform 240 may be more flexible, to allow for greater bending around a surface. Using various combinations of dimensions and materials, rongeur 290 may be configured to cut any of a number of tissues in any of a number of locations in the body.

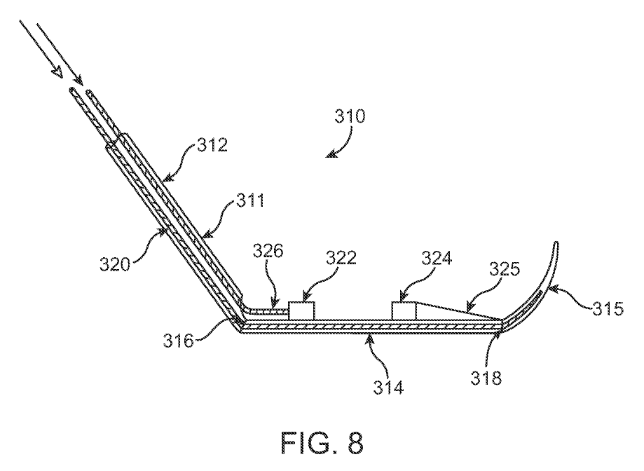

Referring now to FIG. 8, another embodiment of an articulating rongeur 310 is shown in cross-section. Articulating rongeur 310 (of which only a portion is shown) may include a shaft 311 having a proximal shaft portion 312, a distal shaft platform 314 (or “substrate” or “extension”), and an articulation feature 316. Shaft 311 may also include an additional articulation feature 318 and a distal tip 315. Rongeur 310 may also include a proximal blade 322, slidably disposed on platform 314 and coupled with a blade actuating wire 326 that extends through proximal shaft portion 312 and out an aperture therein. A distal blade 324 may be fixedly attached to platform 314, and a tissue capture member 325 may be disposed between distal blade 324 and platform 314 to capture cut tissue that passes under blade 324. Rongeur 310 may further include one or more compression members 320, as described above in reference to FIGS. 6A and 6B. Compressive force may be applied to compression member 320 (hollow-tipped arrow) to articulate rongeur 310 about articulation feature 316, and blade articulating wire 326 may be advanced to advance proximal blade 322 (solid-tipped arrows) to cut tissue.

In the embodiment of FIG. 8, compression member 320 extends through proximal shaft portion 312, through distal platform 314, and into distal tip 315. When compressive force is applied to compression member 320, the force is transmitted all the way to distal tip 315, so that rongeur articulates both at articulation feature 316 and at additional articulation feature 318. In some embodiments, it may be possible to articulate rongeur incrementally, such as by articulating in a first increment at articulation feature 316 and in a second increment at additional articulation feature 318. It may also be possible, in some embodiments, to apply sufficient compressive force to compression member 320 to bend or curl distal tip 315, as shown in FIG. 8. Such bending may facilitate curving rongeur 310 around a curve tissue surface, for example. As described above, in some embodiments, compressive force may also act to bend distal platform 314.

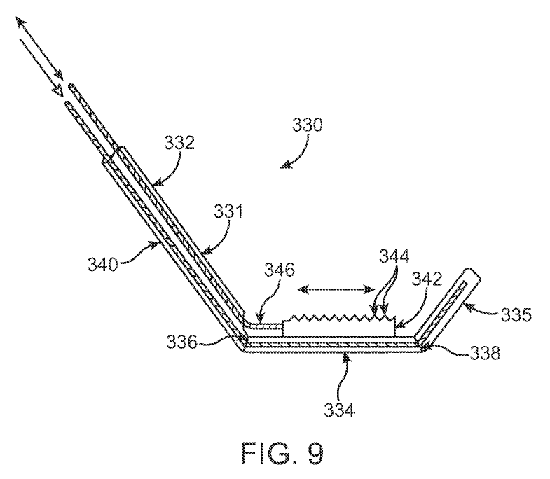

Referring now to FIG. 9, in one embodiment, an articulating tissue cutting device 330 may suitably include a shaft 331 having a proximal portion 332, a distal portion 334 including a distal tip 335, a first articulation feature 336 and a second articulation feature 338. Device 330 may further include a powered reciprocating file 342 having multiple tissue cutting elements 344 and coupled with a drive mechanism 346. A compressive member 340 may be disposed through and attached to shaft 331 for applying compressive force (hollow-tipped arrow) to articulate shaft 331 at articulation features 336, 338.

Shaft 331 and compressive member 340 may have any of the features described above in relation to alternative embodiments. Powered reciprocating file 342 may comprise any suitable reciprocating file device, such as those known in the art and any reciprocating files invented in the future. Generally, file 342 may be reciprocated back and forth (solid, double-headed arrows) by drive mechanism 346 while device 330 is pulled back to urge cutting elements 344 against target tissue, so that cutting elements 344 cut tissue. In some embodiments, cutting elements 344 may open into a collection chamber or area in distal portion 334, where cut tissue may be collected and/or transported proximally through shaft 331 and out of device 330.

In various embodiments, file 342 and drive mechanism 346 may take any of a number of different forms. Various powered reciprocating file devices are described, for example, in U.S. patent application Ser. No. 11/406,486 (Original Attorney Docket No. 78117-200501), titled “Powered Tissue Modification Devices and Methods,” and filed Apr. 17, 2006, the full disclosure of which is hereby incorporated by reference. In one embodiment, reciprocating file 342 may comprise a file such as that invented by Richard J. Harp, founder of SurgiFile, Inc. (The SurgiFile device is described, for example, in U.S. patent application Ser. No. 11/259,625 (Pub. No. 2006/0161189), the full disclosure of which is hereby incorporated by reference). By including one or more articulation features 336, 338 in shaft 331, reciprocating surgical file device 330 may have enhanced ability to reach one or more difficult to reach anatomical areas and/or to gain leverage against one or more structures to facilitate urging file 342 against target tissue.

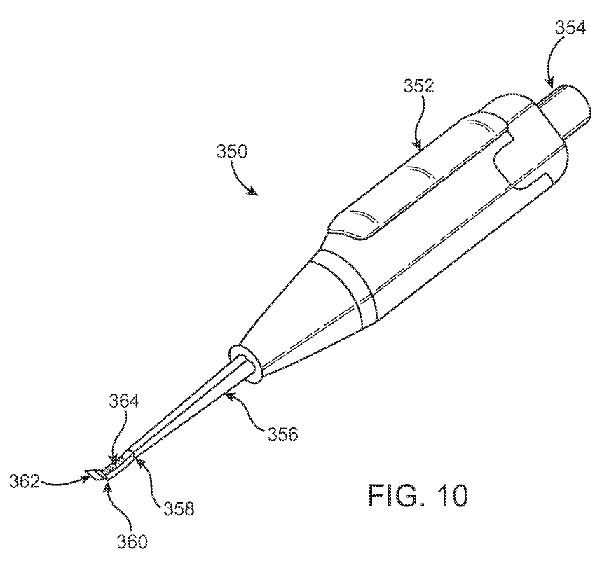

With reference now to FIG. 10, in one embodiment, an articulating reciprocating file tissue cutting device 350 may include a handle 352 with a power source connector 354, a shaft 356 having a first articulation feature 358, a second articulation feature 360 and a distal tip, and a reciprocating file 364. The various portions of shaft 356 may have any of the features described above in relation to various alternative embodiments. An alternative embodiment of device 350 may include only one articulation feature 358, 360, rather than two. Otherwise, device 350 may include any of the features described in U.S. patent application Ser. No. 11/259,625, which was previously incorporated by reference.

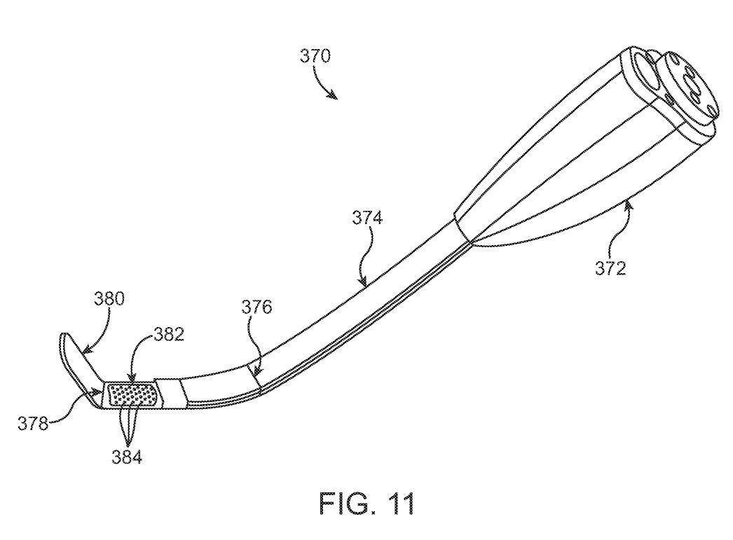

FIG. 11 shows a distal portion of another alternative embodiment of an articulating reciprocating file tissue cutting device 370. In one embodiment, device 370 may include a handle connector 372, a shaft 374 including a first articulation feature 376, a second articulation feature 378 and a distal tip 380, and a reciprocating file 382 having multiple tissue cutting elements 384. As with the previous embodiment, shaft 374 may have any of the various features described above in relation to other embodiments, and device 370 may have any of the features described in U.S. patent application Ser. No. 11/259,625, which was previously incorporated by reference.

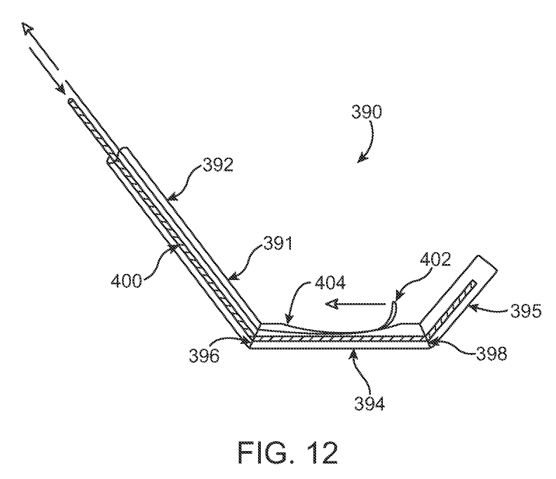

Referring now to FIG. 12, in another embodiment, an articulating tissue cutting device 390 may include a shaft 391 having a proximal portion 392, a distal portion 394, a distal tip 395, a first articulation feature 396 and a second articulation feature 398. A compression member 400 may be disposed through shaft 391 to articulate shaft 391 at articulation features 396, 398. An electrosurgical tissue cutting member 402 may extend through shaft 391 and protrude through (or be exposed through) a window 404 on distal portion 394. Tissue cutting member 402, for example, may comprise a radiofrequency (RF) device, such as a monopolar or bipolar electrosurgical device. In one embodiment, tissue cutting member 402 may be configured as a wire loop. Tissue cutting member 402 may be advanced out of window 404, activated with RF energy, and then retracted (hollow-tipped arrow) to cut tissue, such as ligamentum flavum tissue in the spine or other soft tissue. Further details of such RF tissue cutting devices are provided in U.S. patent application Ser. No. 11/405,848, which was previously incorporated by reference. In one embodiment, tissue cut by tissue cutting member 402 may fall into a tissue collection chamber or hollow area in shaft distal portion 394.

In other alternative embodiments of an articulating tissue cutting device, any of a number of other tissue cutting mechanisms may be used. Exemplary embodiments described above include bladed cutters, reciprocating files, and RF wire cutters, but any other suitable tissue cutting member (or members) may be included in alternative embodiments. For example, tissue cutting members may include but are not limited to blades, abrasive surfaces, files, rasps, saws, planes, electrosurgical devices, bipolar electrodes, monopolar electrodes, thermal electrodes, cold ablation devices, rotary powered mechanical shavers, reciprocating powered mechanical shavers, powered mechanical burrs, lasers, ultrasound devices, cryogenic devices, and/or water jet devices.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. These and many other modifications may be made to many of the described embodiments. Therefore, the foregoing description is provided primarily for exemplary purposes and should not be interpreted to limit the scope of the invention as it is set forth in the claims.

We claim:

1. A device for cutting ligament and/or bone tissue in a lateral recess and/or an intervertebral foramen of a spine of a patient to treat spinal stenosis, the device comprising:
an elongate shaft having a rigid proximal portion and a distal portion articulatable relative to the proximal portion;
a handle coupled with the proximal portion of the shaft;
a tissue cutter disposed on one side of the distal portion of the shaft;
a first actuator coupling the handle with the tissue cutter for activating the tissue cutter to cut tissue; and
a second actuator coupling the handle with the distal portion for articulating the distal portion relative to the proximal portion.

2. A device as in claim 1, wherein the distal portion of the shaft is configured to pass at least partway into an intervertebral foramen of the patient's spine.

3. A device as in claim 1, wherein the distal portion of the shaft is rigid.

4. A device as in claim 1, wherein the distal portion of the shaft is configured to articulate toward the side on which the tissue cutter is disposed.

5. A device as in claim 1, further comprising an articulation member disposed along the shaft between the proximal and distal portions.

6. A device as in claim 5, wherein the articulation member is selected from the group consisting of slits, grooves, hinges and joints.

7. A device as in claim 5, wherein the articulation member comprises:
a first material disposed on the side of the shaft on which the tissue cutter is disposed; and
a second material disposed on an opposite side of the shaft, wherein the first material is more compressible than the second material.

8. A device as in claim 1, wherein the distal portion of the shaft is configured to articulate incrementally from a relatively unflexed position to a first flexed position and to at least a second flexed position.

9. A device as in claim 1, further comprising a locking mechanism coupled with the at least part of the device for locking the distal portion in an articulated position relative to the proximal portion.

10. A device at in claim 1, wherein the tissue cutter is selected from the group consisting of blades, abrasive surfaces, files, rasps, saws, planes, electrosurgical devices, bipolar electrodes, monopolar electrodes, thermal electrodes, cold ablation devices, rotary powered mechanical shavers, reciprocating powered mechanical shavers, powered mechanical burrs, lasers, ultrasound devices, cryogenic devices, and water jet devices.

11. A device as in claim 10, wherein the tissue cutter comprises a translatable blade, wherein the blade has a height greater than a height of a portion of the shaft immediately below the blade, and wherein a total height of the blade and the portion of the shaft immediately below the blade is less than a width of the portion of the shaft immediately below the blade.

12. A device as in claim 11, wherein the tissue cutter further comprises a fixed blade fixedly attached to the shaft, wherein the translatable blade moves toward the fixed blade to cut tissue.

13. A device as in claim 11, wherein the tissue cutter further comprises a fixed backstop fixedly attached to the shaft, wherein the translatable blade moves toward the fixed backstop to cut tissue.

14. A device as in claim 1, wherein the second actuator comprises:
a tensioning wire extending from the handle to the distal portion of the shaft; and
a tensioning member on the handle coupled with the tensioning wire and configured to apply tensioning force to the wire.

15. A device as in claim 1, wherein the second actuator comprises:
a compression member extending from the handle to the distal portion of the shaft; and
a force application member on the handle coupled with the compression member and configured to apply compressive force to the compression member.

16. A device as in claim 15, wherein the compression member is selected from the group consisting of wires, substrates and fluids.

17. A device as in claim 1, wherein the shaft further includes a distal tip articulatable relative to the distal portion of the shaft, wherein the second actuator extends to the distal tip.

18. A device as in claim 1, wherein the first and second actuators are selected from the group consisting of triggers, squeezable handles, levers, dials, toggle clamps, toggle switches and vice grips.

19. A device for cutting tissue in a human body, the device comprising:
an elongate shaft having a rigid proximal portion and a distal portion articulatable relative to the proximal portion;
a handle coupled with the proximal portion of the shaft;
a translatable blade slidably disposed on one side of the distal portion of the shaft;
a first actuator coupling the handle with the tissue cutter for activating the tissue cutter to cut tissue;
a second actuator coupling the handle with the distal portion for articulating the distal portion relative to the proximal portion; and
a locking mechanism configured to lock the distal portion in an articulated configuration relative to the proximal portion.

20. A device as in claim 19, wherein the translatable blade has a height greater than a height of a portion of the shaft immediately below the blade, and wherein a total height of the blade and the portion of the shaft immediately below the blade is less than a width of the portion of the shaft immediately below the blade.

21. A device as in claim 19, wherein the distal portion of the shaft is rigid.

22. A method for cutting ligament and/or bone tissue in a lateral recess and/or an intervertebral foramen of a spine of a patient to treat spinal stenosis, the method comprising:
advancing a distal portion of a tissue cutting device into an epidural space of the patient's spine;
articulating the distal portion relative to a proximal portion of the device;
advancing the distal portion at least partway into an intervertebral foramen of the spine;
urging a tissue cutter disposed on one side of the distal portion of the device against at least one of ligament or bone tissue in at least one of the lateral recess or the intervertebral foramen; and
activating the tissue cutter to cut at least one of the ligament or bone tissue.

23. A method as in claim 22, wherein advancing the distal portion comprises advancing through an access conduit device.

24. A method as in claim 23, wherein the distal portion is advanced through the conduit device and between two adjacent vertebrae into the epidural space without removing vertebral bone.

25. A method as in claim 22, wherein articulating comprises applying tensioning force to a tensioning member disposed longitudinally through the device from the proximal portion to the distal portion.

26. A method as in claim 22, wherein articulating comprises applying compressive force to a compressive member disposed longitudinally through the device from the proximal portion to the distal portion.

27. A method as in claim 22, wherein articulating comprises:
articulating to a first articulated configuration before advancing the distal portion into the foramen; and
further articulating to a second articulated configuration after advancing the distal portion at least partway into the foramen.

28. A method as in claim 22, further comprising locking the distal portion in an articulated position relative to the proximal portion before urging the tissue cutter against tissue.

29. A method as in claim 28, further comprising:
unlocking the distal portion;
straightening the distal portion relative to the proximal portion; and
removing the tissue cutting device from the patient.

30. A method as in claim 22, wherein urging the tissue cutter against tissue comprises applying force to a handle of the tissue cutting device.

31. A method as in claim 22, wherein activating the tissue cutter comprises activating a device selected from the group consisting of blades, abrasive surfaces, files, rasps, saws, planes, electrosurgical devices, bipolar electrodes, monopolar electrodes, thermal electrodes, cold ablation devices, rotary powered mechanical shavers, reciprocating powered mechanical shavers, powered mechanical burrs, lasers, ultrasound devices, cryogenic devices, and water jet devices.

32. A method as in claim 31, wherein activating the tissue cutter comprises advancing a translatable blade toward one of a stationary blade and a backstop.

33. A method as in claim 31, wherein activating the tissue cutter comprises retracting a translatable blade toward one of a stationary blade and a backstop.

34. A method as in claim 31, wherein activating the tissue cutter comprises translating two blades toward one another.
